(19) United States
(12) Patent Application Publication
(10) Pub. No.: US 2017/0014374 A1
(43) Pub. Date: Jan. 19, 2017

(54) COMPOSITION FOR TREATMENT OF DYSLIPIDEMIA AND INFLAMMATION

(71) Applicant: THORNE RESEARCH, INC., Sandpoint, ID (US)

(72) Inventors: Bodi Zhang, New York City, NY (US); Michael A Schmidt, Fort Collins, CO (US); Mark Houston, Nashville, TN (US); Robert Rountree, Boulder, CO (US)

(73) Assignee: THORNE RESEARCH, INC., Sandpoint, ID (US)

(21) Appl. No.: 14/798,032

(22) Filed: Jul. 13, 2015

(51) Int. Cl. A61K 31/352; A61K 31/12; A61K 9/00; A61K 36/82

(52) U.S. Cl. CPC A61K 31/352; A61K 36/82; A61K 31/12; A61K 9/0053

(57) ABSTRACT

The present invention provides compositions that are substantially niacin free for treating dyslipidemia and/or inflammation in a subject. Compositions of the invention include a therapeutically effective amount of a mixture phytosomes obtained from quercetin and at least one other phytosome obtained from the group consisting of curcurmin, green tea, and a combination thereof. The present invention also provides a method for treating dyslipidemia and/or inflammation in a subject.

### FIELD OF THE INVENTION

The present invention relates to a composition for treatment of dyslipidemia and/or inflammation without the use of a statin medication. In particular, compositions of the invention are based on a mixture of phytosomes obtained from at least two, typically at least three botanicals. The composition of the invention includes phytosomes obtained from quercetin and curcurmin. It can also include phytosomes obtained from Green Tea. The invention also relates to treating dyslipidemia and/or inflammation using the composition of the invention.

### BACKGROUND OF THE INVENTION

Dyslipidemia currently afflicts more than 30 million people in the United States. While there are medications available, the majority of dyslipidemia medications are HMG-CoA reductase inhibitors. These statin medications, all of which are synthetic drugs, have many adverse side effects such as anemia, acidosis, cataracts, diabetes, immune depression, memory loss, pancreas dysfunction, liver dysfunction, chronic inflammation, polyneuropathy and rhabdomyolysis.

Therefore, there is a need for treating dyslipidemia without using a statin medication or a HMG-CoA reductase inhibitor or a synthetic drug.

### SUMMARY OF THE INVENTION

The present invention provides a composition that is based on botanicals that can be used to treat dyslipidemia. Surprisingly and unexpectedly, it has been found that the composition of the invention can also treat inflammation. Accordingly, the composition of the invention can be used to treat dyslipidemia and/or inflammation.

In one aspect, the composition of the invention comprises a therapeutically effective amount of a mixture of phytosomes. In one embodiment, the mixture of phytosomes include a phytosome obtained from quercetin and at least on other phytosome obtained from the group consisting of curcurmin, green tea, and a combination thereof. The composition of the invention can also include a therapeutically effective amount of a lipid-modifying composition.

The invention also provides a method for treating dyslipidemia and/or inflammation in a subject.

### BRIEF DESCRIPTION OF THE DRAWINGS

FIG. 1 is a graph of the control group vs. the treatment group for the total cholesterol level.

FIG. 2 is a graph of the control group vs. the treatment group for the total LDL-cholesterol level.

FIG. 3 is a graph of the control group vs. the treatment group for the total VLDL-c numbers level.

FIG. 4 is a graph of the control group vs. the treatment group for the total LDL-P numbers level.

FIG. 5 is a graph of the control group vs. the treatment group for the total non-HDL particle numbers level.

FIG. 6 is a graph of the control group vs. the treatment group for the total HDL particle numbers level.

FIG. 7 is a graph of the control group vs. the treatment group for the total ApoB level.

FIG. 8 is a graph of the control group vs. the treatment group for the total triglyceride level.

FIG. 9 is a graph of the control group vs. the treatment group for the total oxLDL level.

### DETAILED DESCRIPTION OF THE INVENTION

Some aspects of the invention provide a composition that is botanically derived that can be used to treat dyslipidemia and/or inflammation in a subject. “Treating” or “treatment” of a disease includes: (1) preventing the disease, i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms. No conventional drugs to date are known to simultaneously treat dyslipidemia while lowering inflammation biomarkers.

The present inventors have discovered that compositions of the invention can be used to treat dyslipidemia and/or inflammation simultaneously. In particular, it was discovered that compositions of the invention are effective in treating dyslipidemia as well as lowering inflammatory biomarker levels. In one particular aspect of the invention, the composition comprises a therapeutically effective amount of a mixture of phytosomes. “Pytosome” is a complex of a natural active ingredient and a phospholipid. The term “natural active ingredient” refers to an ingredient that is found in a natural source, typically a botanical source that has therapeutic or biologically active activity. In the composition of the invention, therapeutic or biological activity of “natural active ingredient” refers to ability to modulate biomarkers related to dyslipidemia and/or inflammation. Accordingly, compositions of the invention are directed to those that can be used to treat dyslipidemia and/or inflammation in a subject, typically a mammal, and often human. Exemplary phospholipids that can be used to produce phytosomes of the invention include, but are not limited to, soy lecithin, egg lecithin, sunflower lecithin, and other phospholipids known to one skilled in the art.

The composition of the invention is substantially niacin (i.e., vitamin B₃ or nicotinic acid) free. The term “niacin free” means that niacin is not present in any significant amount as to affect the biological activity. While some trace amount of niacin may be present inadvertently, e.g., as a part of a natural (e.g., botanical) source from ingredients used in compositions of the invention, no amount of niacin is intentionally added. Alternatively, the term “niacin free” means the composition of the invention has no more than 1% by weight (relative to the total weight of the composition), typically no more than 0.5% by weight, often no more than 0.1% by weight, more often no more than 0.05% by weight, and most often no more than 0.01% by weight of niacin.

Compositions of the invention can also include a therapeutically effective amount of a lipid-modifying composition. Lipid-modifying composition refers to a composition that includes a lipid-modifying agent. Lipid-modifying agents are materials that can modulate the lipid profile of the subject. Exemplary lipid-modifying agents useful in compositions of the invention include, but are not limited to, phytosterol esters, green tea, curcumin, luteolin, quercetin, aged garlic, red yeast rice, N-acetyl-L-cysteine, berberine, licorine and lycopene. In one particular embodiment, the lipid-modifying composition in compositions of the invention include, but are not limited to, green tea, curcumin, luteolin, quercetin, aged garlic, red yeast rice, N-acetyl-L-cysteine, berberine, licorine, Lycopene, and a combination thereof. When present, the amount of lipid-modifying composition can be about 80% by weight or less, typically about 70% by weight or less, often about 60% by weight or less, and most often about 50% by weight or less relative to the total weight of the composition. In one particular embodiment, the amount of lipid-modifying composition is from about 1% to about 50% by weight with the remainder being a mixture of phytosomes and any excipients and/or carriers that are used in the formulation.

In another embodiment, the amount of phytosome mixture in compositions of the invention is about 90% or less, typically about 80% or less and often about 75% or less relative to the total weight of the composition. In one particular embodiment, the amount of phytosome mixture ranges from about 1% to about 80% with the remainder being the lipid-modifying composition and/ro any excipients and/or carriers that are used in the formulation.

Generally, biologically active ingredients in compositions of the invention are obtained from botanical sources. In one particular embodiment, the mixture phytosomes include phytosomes obtained from quercetin and at least one other phytosome obtained from the group consisting of curcurmin, green tea, and a combination thereof. Quercetin phytosomes can be produced by obtaining Sophora japonica extract (e.g., extracts obtained from leaf) and combining the extract with a phosphatidylcholine complex. Typically, the phosphatidylcholine complex used in producing a phytosome is lecithin. While any source of lecithin can be used in producing a phytosome, generally lecithin obtained from soy, egg, sunflower or a combination thereof is used. The amount of phosphatidylcholine complex used in producing a phytosome can range from 1% by weight to about 80% by weight relative to the amount of botanical extract. Typically, the amount of phosphatidylcholine complex used in producing a phytosome ranges from about 5% by weight to about 60% by weight, often about 10% by weight to about 45% by weight, and most often from about 15% by weight to about 40% by weight. However, it should be appreciated that the amount of phosphatidylcholine complex in a phytosome is not limited to these ranges. The amount of quercetin phytosome in composition of the invention can range from about 10 mg/unit to about 500 mg/unit, and typically from about 75 mg/unit to about 200 mg/unit. However, it should be appreciated that the range can vary depending on a particular results desired in treating dyslipidemia.

Curcumin phytosomes can be obtained from curcuma longa extract. For example, root extract of curcuma longa can be combined with a phosphatidylcholine complex to produce the phytosome. The amount and the nature of phosphatidylcholine complex used are those described above. The amount of curcumin phytosome in composition of the invention can range from about 10 mg/unit to about 500 mg/unit and typically from about 20 mg/unit to about 400 mg/unit. However, it should be appreciated that the range can vary depending on a particular results desired in treating dyslipidemia. Alternatively, the amount of curcumin phytosome relative to quercetin phytosome ranges from about 1 to 25:1 and typically from about 10:1, and often from about 1:1.

In some embodiments, compositions of the invention include a mixture of quercetin phytosome and curcurmin phytosome.

Still in other embodiments, the composition of the invention includes at least three different botanical phytosomes. In particular, in specific embodiment includes quercetin phytosome, curcurmin phytosome, and green tea phytosome. Any green tea known to one skilled in the art can be used to produce green tea phytosome of the invention. In one particular embodiment, Camellia sinensis extract, typically leaf extract thereof, is used to produce the green tea phytosome of the invention. Again, the amount and the nature of phosphatidylcholine complex used are described above. The amount of green tea phytosome in composition of the invention can range from about 10 mg/unit to about 500 mg/unit and typically from about 20 mg/unit to about 400 mg/unit. However, it should be appreciated that the range can vary depending on a particular results desired in treating dyslipidemia. Alternatively, the amount of green tea phytosome relative to quercetin phytosome ranges from about 25:1, typically from about 10:1, and often from about 1:1. Throughout this disclosure, the term “about” when referring to a number or a particular value means ±20%, typically ±10%, often ±5%, and most often ±1%.

It should be appreciated that some of the phytosomes in compositions of the invention can also have lipid-modifying activity and as such, the amount disclosed herein refers to the total amount of such phytosome(s) in the composition.

Compositions of the invention are typically formulated for oral administration. However, it should be appreciated other means of administration, e.g., intravenous, can also be used. Typically, the composition formulated as a soft or hard gelatin, capsule, tablet, powder, or in a liquid form such as beverage. While the composition can be formulated for a multiple dosage regime, it is generally formulated as a single dosage unit or a twice daily dosage unit. As used herein, the term “unit” refers to a single tablet, capsule, gelatin, or any other single article of formulation.

It should be appreciated that other phospholipid source similar to soy, egg, or sunflower can also be used. Accordingly, the scope of the invention is not limited to any particular phospholipid source disclosed herein but includes any phospholipid source that are known to one skilled in the art or are conventionally available as well as ones that are developed hereafter and are compatible with the phytosomes disclosed.

In some embodiments, the composition of the invention is administered to the subject such that 500 mg per day of each phytosome is provided to the subject. Such administration can be divided into 2 doses per day. Such amount is typically provided in capsules comprising 250 mg per dosage unit (e.g., capsule) of each phytosome. Compositions of the invention can be administered orally, typically with meals.

Compositions of the invention are useful in treating dyslipidemia and/or inflammation. As discussed in detail below, compositions of the invention has modulating affect on various biomarkers related to dyslipidemia and inflammation. Exemplary biomarkers that are affected by compositions of the invention include, but are not limited to, Platelet aggregation, TNFα, IL-1β, IL-2, IL-4, IL-6, IL-10, Fibrinogen, NFκB, MucA, MucB, ICAM-1, VCAM1, VEGF, MCP-1, E-selectin, uPA, Malondialdehyde, F2-Isoprostanes (8-epi-PGF2α), LDL oxidizability, Homocysteine, PPARα, PPARγ, PPARγ, TGFδ, Collagen-4, IL-17, MMP, LTB4, PGE2, MAP, Cholesterol Balance, Total Cholesterol, Direct LDL, HDL, Triglycerides, Non-HDL, APOB, LDL-p, sdLDL, VLDL, Lp(a), ApoA-1, Fibrinogen, hsCRP, LpPLA2, ApoE, MPO, HbA1C, Glucose, Insulin resistance, F2 isoprostanes, Galectin 3, Serum Coenzyme Q10, etc.

In formulating compositions of the invention in a solid form, e.g., a tablet form, typically a tablet-pressing (i.e., compressing) process is used. Such a process generally involves blending all ingredients as dry, powdered or granular solids. These solids typically of relatively uniform in particle size and freely flowing. An inactive ingredient (i.e., excipient) such as a binder is typically added to help hold the tablet or caplets or capsules together and give it strength. A wide variety of binders can be used. Exemplary binders include, but are not limited to, sucrose, corn starch, pregelatinized corn starch, microcrystalline cellulose and modified cellulose (e.g., hydroxypropyl methylcellulose, ethylcellulose), polyvinylpyrrolidone (PVP), hydroxypropyl cellulose), and the like. An ingredient that acts as a disintegrant to aid tablet dispersion once swallowed can also be added. Such ingredients aid in releasing the therapeutically active components for absorption. Some binders, such as many forms of starch and cellulose, are also excellent disintegrants. An exemplary disintegrant includes, but is not limited to, sodium croscarmellose. Small amounts of lubricants are also typically added. Exemplary lubricants include, but are not limited to, magnesium stearate, calcium stearate, stearic acid (stearin), hydrogenated oil, and sodium stearyl fumarate. Once pressed, the lubricant helps the tablets inter alia to be more easily ejected from the die.

While tablets can be made in virtually any shape, typically they are round, oval or capsule shaped. Tablet diameter and shape are generally determined by the machine tooling used to produce them, which typically consists of a die, and an upper and a lower punch. The thickness of the tablet is generally determined by the amount of tablet material and the position of the punches in relation to each other during compression (i.e., compression force). Generally, the greater the pressure applied during compression the harder and thinner the tablet. Tablets should be hard enough that they don't break up in the bottle and yet should readily disintegrate in the gastric tract. Tablets also need to be strong enough to resist the stresses of packaging, shipping and handling.

Compositions of the invention can also include other nutritional supplement components, e.g., iron, vitamin B₁₂, folic acid, vitamin D₃, coenzyme Q₁₀, β-carotene, ascorbic acid, etc. along with binders, fillers, lubricants, disintegrants, etc. and compressed to a desired size to form a tablet.

The tablet or any solid formulation can optionally be coated. Typically a polymer coating is applied to make the solid formulation smoother and easier to swallow, to control the release rate, to make it more resistant to the environment (e.g., extending its shelf life), or to enhance the tablet's appearance. There are many ways to coat tablets that are known to one skilled in the art. Tablets are typically coated with sugar or with a polymer and/or a polysaccharide. In some embodiments, a sub-coating is applied to the tablet, for example, a mixture of an isopropyl alcohol solution of flavoring agent (e.g., oil orange and vanilla), and an aqueous solution of Opadry® clear (from Colorcon) is used to subcoat the tablet. The final coating is then applied to the tablet, for example, by using an aqueous solution of colored Opadry® II.

Compositions of the invention can also include other desirable additives such as, but not limited to, starches, sugars, fats, antioxidants, amino acids, proteins, derivatives thereof or combinations thereof. Antioxidants generally improve the stability of the final composition. Inclusion of additives that assist in formulating the final composition are also desirable.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### EXAMPLES

Study Objective: The purpose of this study was to evaluate the efficacy and side effects of the composition of the invention. The composition of the invention comprises natural compounds for treating dyslipidemia. This study was conducted over 4 months, in adult subjects not taking lipid lowering drugs or lipid lowering nutraceutical supplements. The composition of the invention for the study had the following ingredients in one pack: phytosterol esters 800 mg, aged garlic extract (bulb) (Allium sativum) (Kyolic®) 600 mg, red yeast rice (monascus purpureus) 500 mg, curcumin phytosome (curcuma longa extract (root)/phosphatidylcholine complex), 250 mg, green tea phytosome (camellia sinensis extract (leaf)/phosphatidylcholine complex) 250 mg, N-acetyl-L-cysteine 250 mg, berberine HCl (from Indian barberry extract) (roo) (Berberis aristata) 200 mg, deglycyrrhizinated licorice (DGL) extract (root) (Glycyrrhiza spp.) 100 mg, trans-resveratrol 40 mg, quercetin phytosome (sophora japonica concentrate (leaf)/phosphatidylcholine complex from sunflower) 50 mg, lycopene (from tomato) (lycopersicon esculentum) 4 mg. Other ingredients included excipients and carrier such as microcrystalline cellulose, hypromellose (derived from cellulose) capsule, leucine, silicon dioxide, calcium laurate, gelatin (bovine), purified water, glycerin (from vegetable source) and turmeric (color) gelcap, medium chain triglyceride oil, lecithin (sunflower). Patients took two packs per day.

A double-blind randomized placebo-controlled prospective study was conducted with 40 subjects ages 18 to 80 years with fasting dyslipidemia (LDL 130-250 mg %) based on initial screening with direct LDL measurements. Primarily recruited subjects were statin-intolerant or refuse to take statins or any other lipid lowering drug for any reason. In addition, the subjects with any of the following criteria were excluded from this study: (1) myocardial infarction, CABG, PCTA or stent placement surgery within 5 years, known clinical CHD symptoms or clinical angina; (2) history of cerebrovascular accident (CVA); (3) creatinine over 2.5 mg %; (4) known allergy or sensitivity to any components of the composition of the present invention; (5) chronic liver disease with AST, ALT, alkaline phosphatase over 1.5× normal; (6) known cancer within 2 years; (7) clinical congestive heart failure (systolic or diastolic CHF); (8) type I or Type 2 diabetes mellitus; (9) pregnant or nursing women; and (10) women of child bearing age that are not on approved and accepted contraception.

The protocol for this study included obtaining informed consent prior to the enrollment in the study. Subject's each visit included the measurements of blood pressure, heart rate, waist circumference, body weight and body composition with BIA. For each subjects, blood samples were drawn for laboratory analysis after a 12 hour fast, except for water and regular medications where were allowed to be taken prior to each visit. Laboratory analysis of the blood sample were done at baseline and at variable times. The subjects were seen in the mornings in a fasting state at monthly intervals. Patients were advised to maintain present weight, nutrition program and diet, exercise regimen, alcohol intake, smoking, caffeine intake and prescription medications (excluding any lipid lowering agent) during the study. For the duration of the study, subjects were prohibited from taking medications that are known to effect serum lipids. Any nutritional supplement known to effect lipids will not be permitted. Subjects that wanted to participate in this study were also advised to discontinue use of such medications or supplements at least one month prior to study initiation.

ENDOPAT testing for endothelial function, heart rate variability (HRV) and augmentation index (AI) were done at baseline and at 2 months and at final visit (3 times total). Each of these visits included blood pressure (BP), weight, waist circumference and BIA and a complete review of clinical aspects and adverse effects.

CAPWA (computerized arterial pulse wave analysis) for arterial compliance and central BP were also done at baseline at 2 months and at final visit. (3 times total)

Patients were administered the composition of the invention capsules or matching placebo twice per day with meals for the duration of the study. Compliance was determined by pill count at each visit, interviews and patient records and forms.

To examine whether the composition of the invention can change the level of biomarkers over time, the adjusted least-squares means of biomarkers were estimated by the two intervention arms (the composition of the invention arm and the placebo arm) using generalized linear models. P-values of the null hypothesis for each biomarker were also determined. All statistical analysis were performed using SAS (Cary, N.C.).

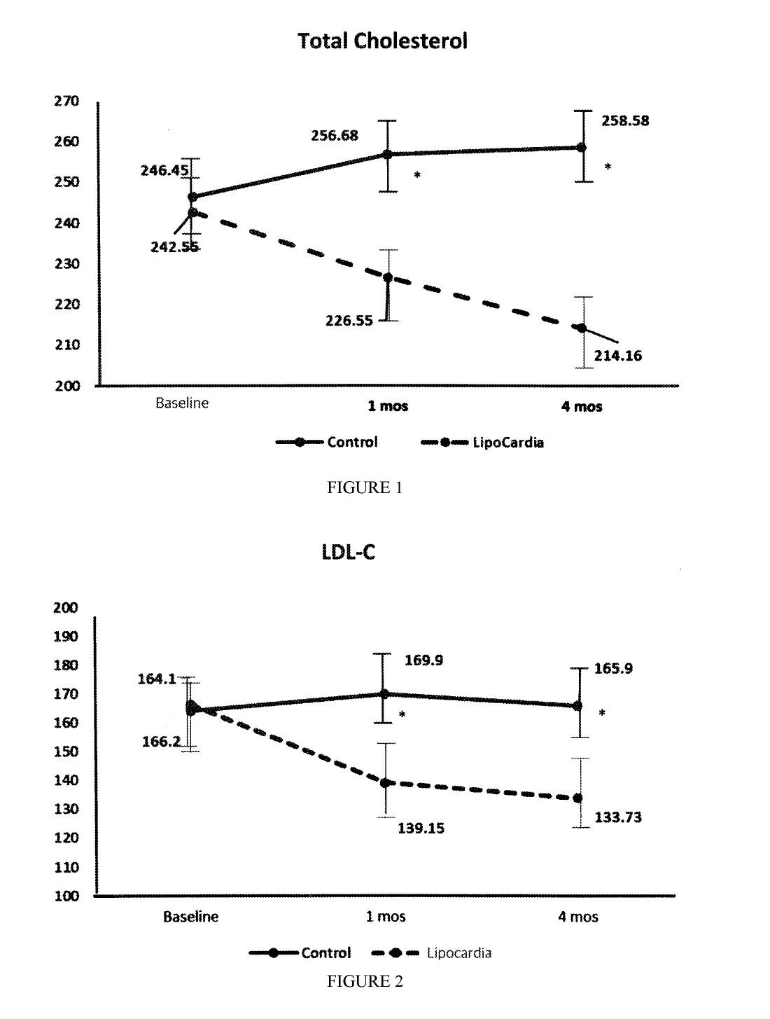

As can be seen in FIG. 1, the composition of the invention significantly reduced total cholesterol level in treatment group compared to placebo group. The total cholesterol level of subjects was measured at baseline, one month and four month timeline. FIG. 1 shows about 19% decrease in the average total cholesterol level in the treatment group (subjects receiving the composition of the invention, p-value=0.0029) compared to the control group (p-value=0.0031).

The total LDL-cholesterol level in treatment group compared to placebo (i.e., control) group is shown in FIG. 2. The total LDL-cholesterol level of subjects was also measured at baseline, one month and four month timeline. FIG. 2 shows about 19% (p-value=0.0099) decrease in the average total LDL-cholesterol level in the treatment group compared to the control group.

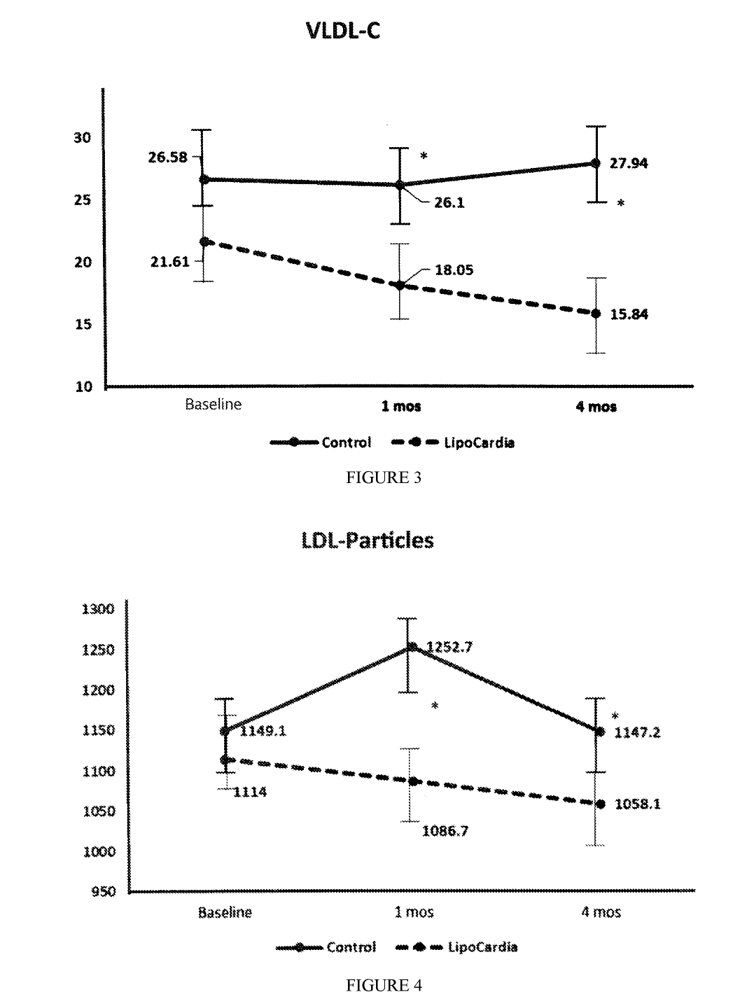

FIG. 3 shows the total VLDL-c numbers level in treatment group compared to control group. The total VLDL-c numbers level of subjects was also measured at baseline, one month and four month timeline. FIG. 3 shows about 43.3% (p-value=0.0057) decrease in the average total VLDL-c numbers level in the treatment group compared to the control group.

As can be seen in FIG. 4, the composition of the invention also significantly reduced the total LDL-P numbers level in treatment group compared to placebo group. The total LDL-P numbers level of subjects was measured at baseline, one month and four month timeline. Referring again to FIG. 4, the average total LDL-P numbers level decreased about 8.9% in the treatment group (p-value=0.0494, standard deviation (SD) for both groups=about 91 mg/dl).

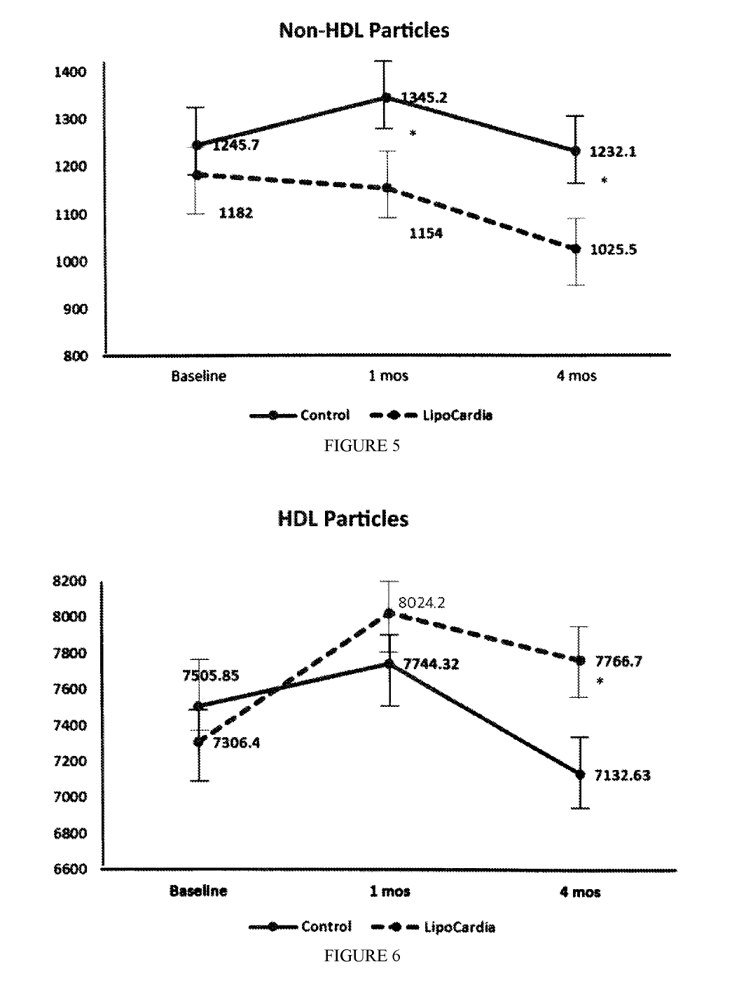

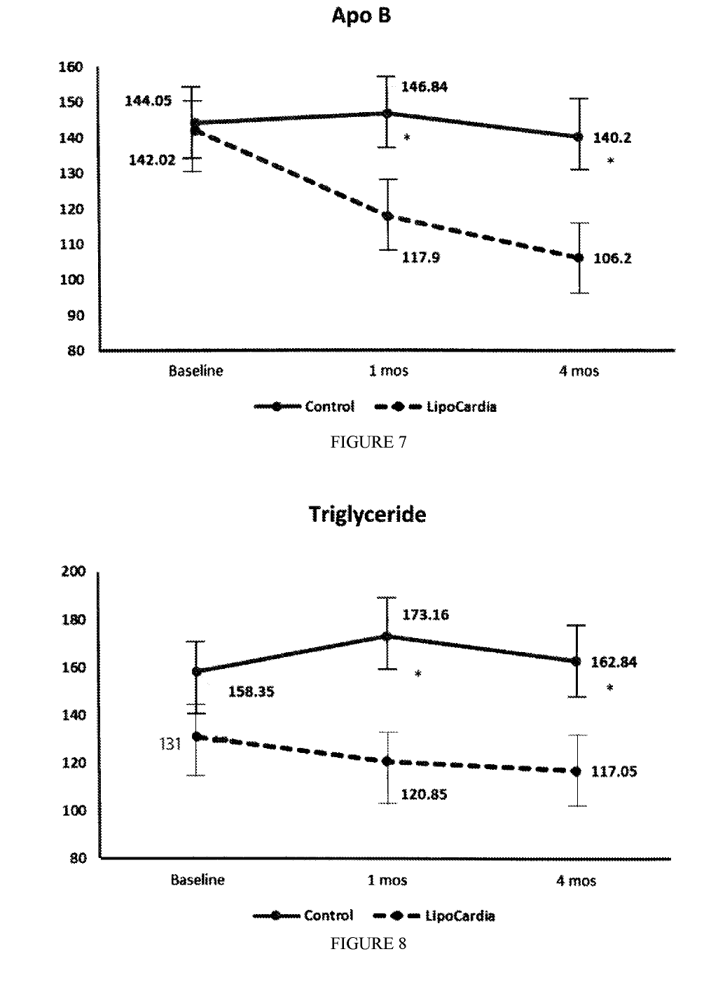

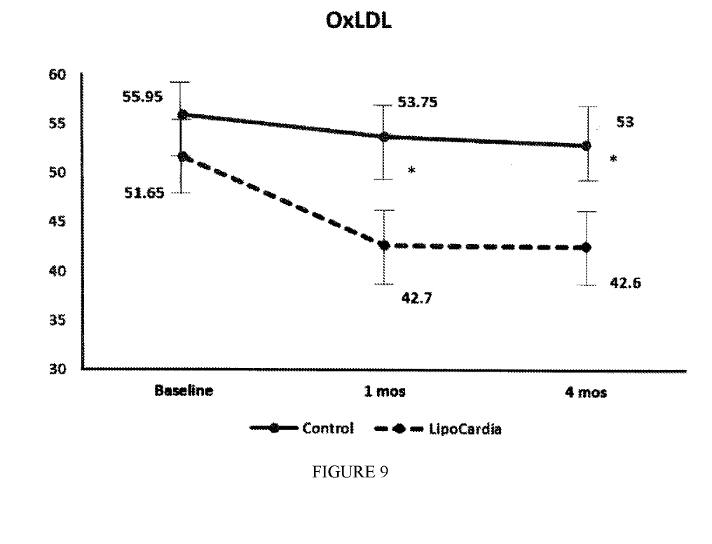

Other biomarkers that were tested include, but not limited to, non-HDL particles (FIG. 5, 17% decrease in the treatment group, p-value=0.041, SD=47 mg/dl); HDL particle numbers (FIG. 6, increase of 8.9% in the treatment group, p-value=0.0183); Apo-B numbers (FIG. 7, 24.2% decrease in the treatment group, p-value=0.0029, SD=about 6.7 mg/dl); triglyceride level (FIG. 8, decrease of 29% in the treatment group, p-value=0.014, SD=about 25 mg/dl); and oxLDL level (FIG. 9, 19.7% decrease in the treatment group, p-value=0.00123, SD=about 2.3 mg/dl).

Surprisingly, unlike most of the stain drugs, the composition of the invention did not significantly reduce CoQ10 level. There was no statistical difference between the treatment and placebo group with regards to CoQ 10 level.

The composition of the invention also had a positive affect on the treatment group for various inflammatory markers. In particular, Pearson analysis of data showed that hs-CRP, one of the major inflammatory markers, was significantly reduced in the treatment group as can be seen in the table below.

| | | Baseline- | Midpoint- | Baseline- |
|---|---|---|---|---|
| | | midpoint | endpoint | endpoint |
| Baseline CRP | Coefficients | −0.9135 | −0.0828 | −0.9203 |
| | P-values | <0.0001 | 0.7694 | <0.0001 |

As can be seen in the above table, hs-CRP was reduced significantly early in the trial (i.e., from baseline to midpoint measurement), and remained reduced at a similar level thereafter (i.e., compare baseline to midpoint vs. baseline to endpoint).

Pearson analysis of another major inflammatory marker TNF also showed a significant reduction in the treatment group as can be seen in the table below.

| | | Baseline- | Midpoint- | Baseline- |
|---|---|---|---|---|
| | | midpoint | endpoint | endpoint |
| Baseline TNFA | Coefficients | −0.8946 | 0.1959 | −0.8531 |
| | P-values | <0.0001 | 0.4216 | <0.0001 |

Again, similar to hs-CRP, TNF was also reduced significantly early in the trial and remained at the reduced level thereafter.

Similarly, as can be seen in the table below Pearson analysis of IL-6, another major inflammatory biomarker, also showed significant reduction early in the trial and remained at a relatively similar level thereafter.

| | | Baseline- | Midpoint- | Baseline- |
|---|---|---|---|---|
| | | midpoint | endpoint | endpoint |
| IL-6 Baseline | Coefficients | −0.9952 | 0.0728 | −0.9915 |
| | P-values | <0.0001 | 0.7672 | <0.0001 |

Another inflammatory biomarker, IL-17, also showed a significant reduction in the treatment group. Pearson analysis of this biomarker showed reduction of about −0.9754 within the first two months and stayed at this reduction level through out the treatment as shown in the table below.

| | | Baseline- | Midpoint- | Baseline- |
|---|---|---|---|---|
| | | midpoint | endpoint | endpoint |
| Baseline | Coefficients | −0.9754 | 0.2211 | −0.9727 |
| | P-values | <0.0001 | 0.4106 | >0.0001 |

Conclusion: As shown above, surprising degrees of LDL, TG, and inflammation marker reduction with concurrent increase of HDL particle number and size in subjects who were administered the composition of the invention was observed. The results were achieved with the composition of the invention, typically where the dosing comprised a single capsule of the composition of the invention taken 2 times per day with meals. The dose of each phytosome was about 500 mg/day.

These subjects, both male and female, ages 18 to 80 years old were primarily statin-intolerant or unwilling to take statins or other lipid lowering medication for any reason. The significant benefits were seen when compared to the placebo group. As discussed above, Pearson analysis on four inflammatory markers, hs-CRP, TNFA, IL-6, and IL-17, all showed a significant decrease within the midpoint of testing and remained at the reduced level throughout the testing period.

Surprisingly, unlike the statin medications, the composition of the invention did not show any significant change in CoQ10. The total cholesterol level in the treatment group dropped 19% compared to the placebo group. LDL-Cholesterol levels in the treatment group also dropped 19% compared to the placebo group. In addition, VLDL-c levels in the treatment group dropped 43.3% compared to the placebo group. And LDL-P numbers in the treatment group dropped 8.9% compared to the placebo group.

Overall, non-HDL particle numbers in the treatment group decreased by 17% compared to the placebo group. Significantly, HDL particle numbers in the treatment group increased by 8.9% compared to the placebo group. ApoB levels in the treatment group decreased by 24.2% compared to the placebo group. Triglyceride (TG) levels in the treatment group decreased by 29% compared to the placebo group. And oxLDL levels in the treatment group decreased by 19.7% compared to the placebo group.

These results clearly indicate, the composition of the invention can be used to treat dyslipidemia and reduce inflammatory biomarkers in subject.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A substantially niacin free composition for treating dyslipidemia and inflammation in a mammal, said composition comprising a therapeutically effective amount of a mixture phytosomes obtained from quercetin and at least one other phytosome obtained from the group consisting of curcurmin, green tea, and a combination thereof.

2. The composition of claim 1, wherein said mixture of phytosomes comprises a phytosomes obtained from quercetin and curcurmin.

3. The composition of claim 1, wherein said mixture of phytosomes is obtained from at least three different botanicals.

4. The composition of claim 3, wherein said mixture of phytosomes comprises phytosomes obtained from quercetin, curcurmin, and green tea.

5. The composition of claim 1, wherein said curcumin phytosome is obtained from Curcuma longa.

6. The composition of claim 1, wherein said green tea phytosome is obtained from Camellia sinensis.

7. The composition of claim 1, wherein quercetin phytosome is obtained from Sophora japonica.

8. The composition of claim 1, wherein said phytosome comprises a phospholipid selected from the group consisting of lecithin obtained from soy, egg, sunflower, or a combination thereof.

9. The composition of claim 8, wherein the amount of phospholipids in said composition ranges from about 10% to about 45%.

10. The composition of claim 1, wherein said composition is formulated as a soft or hard gelatin, capsule, tablet, powder, or beverage.

11. The composition of claim 1 wherein said composition further comprises a therapeutically effective amount of a lipid-modifying composition.

12. The composition of claim 1, wherein the amount of said lipid-modifying composition ranges from about 1% to about 50%.

13. The composition of claim 1, wherein the amount of said mixture phytosomes in said composition ranges from about 1% to about 80%.

14. A method for treating dyslipidemia or inflammation in a subject, said method comprising administering a therapeutically effective amount of a composition of claim 1 to a subject in need of such a treatment.

15. The method of claim 14, wherein said composition is administered orally.
